(19) United States
(12) Patent Application Publication
(10) Pub. No.: US 2008/0051372 A1
(43) Pub. Date: Feb. 28, 2008

(54) METHODS UTILIZING CELL-SIGNALING LYSOPHOSPHOLIPIDS

(75) Inventor: Jerold Chun, La Jolla, CA (US)

Correspondence Address: MICHAEL BEST & FRIEDRICH LLP, ONE SOUTH PINCKNEY STREET, P O BOX 1806, MADISON, WI, 53701, US

(73) Assignee: THE SCRIPPS RESEARCH INSTITUTE, La Jolla, CA (US)

(21) Appl. No.: 11/844,793

(22) Filed: Aug. 24, 2007

Related U.S. Application Data
(60) Provisional application No. 60/823,472, filed on Aug. 24, 2006.

(51) Int. Cl. A61K 31/661; A61P 25/28; C12N 5/02; C12Q 1/02

(52) U.S. Cl. 514/114; 435/29; 435/375; 514/143

(57) ABSTRACT

The invention relates to methods of modulating neurite outgrowth, in culture or in a subject. The methods generally utilize cell-signaling phospholipids which interact and bind to the G protein-coupled cellular receptors (GPCRs). Such phospholipids include lysophospholipids, as well as synthetic lysophospholipid receptor agonists and antagonists that may be chemically distinct from lysophospholipids. The methods include contacting astrocytes with an effective amount of a lysophospholipid agent, and contacting neurons with the astrocytes. The methods also include treating neurons by contacting the neurons with astrocytes pretreated with a lysophospholipid agent. The methods further include contacting the neurons with an effective amount of an astrocyte-derived soluble factor (ADSF).

### CROSS REFERENCE TO RELATED APPLICATIONS

This application claims priority to U.S. Provisional Application No. 60/823,472 filed Aug. 24, 2006, which is incorporated by reference in its entirety.

### STATEMENT REGARDING FEDERALLY SPONSORED RESEARCH AND DEVELOPMENT

This invention was made with United States government support awarded by the following agency: NIMH, Grant No. MH-01723. The United States government has certain rights in this invention.

### INTRODUCTION

Lysophospholipids (LPs), such as lysophosphatidic acid (LPA) and sphingosine 1-phosphate (S1P), are membrane-derived bioactive lipid mediators. LPs affect many biological processes including neurogenesis, angiogenesis, would healing, immunity and carcinogenesis.

LPs have recently been added to the list of intercellular lipid messenger molecules. Their cellular responses are triggered by activation of specific seven-transmembrane domain receptors known as G protein-coupled receptors (GPCRs). LPs interacts with GPCRs, coupling to various independent effector pathways including inhibition of adenylate cyclase, stimulation of phospholipase C, activation of MAP kinases, and activation of the small GTP-binding proteins Ras and Rho. LPA signals cells, in part, via the GPCRs LPA₁, LPA₂, LPA₃, LPA₄ and LPA₅. These receptors generally share 50-55% identical amino acids, although in some instances less, and cluster with 5 other receptors, S1P₁, S1P₂, S1P₃, S1P₄ and S1P₅ for the structurally-related S1P.

LPA receptors are expressed by several neural cell types including neurons, oligodendrocytes, Schwann cells, astrocytes, and microglia. Stimulation of LPA receptors is involved in several developmental processes within the mammalian nervous system such as growth and folding of the cerebral cortex; growth cone retraction, cell survival, cellular migration, cell adhesion and proliferation. These receptor interactions exemplify the relevance of lipid signaling for neural development and function, and underscore the importance of understanding the cellular responses elicited by these ligands under normal and pathological conditions. Surprisingly, there has been lack of information regarding the physiological roles of LPA receptors and their signaling systems in neuron-glia interaction, a crucial caveat for brain development and function.

Neuron-glia interactions play an important role in several processes of brain development such as neurogenesis, neuronal migration; axonal guidance; myelination, synapse formation and glial maturation. Astrocytes, the most abundant glial cell, provide most of the extracellular matrix (ECM) components in the central nervous system (CNS) and are strongly involved in determining neuronal polarity and axonal pathfinding. Further, astrocytes represent a potent source for most neurotrophic factors involved in neuronal proliferation, survival and stem cell fate determination.

LPA elicits a broad spectrum of response in astrocytes such as decrease in glutamate and glucose uptake, stimulation of reactive oxygen species synthesis, increase in intracellular calcium concentrations and modulation of astrocyte proliferation and morphology. Although it is not completely clear which type of LPA receptor is involved in each of these functions, astrocytes have been shown to express all isoforms of LPA receptors in vitro (Steiner et at, Multiple astrocyte response to lysophosphatidic acids, 2002, Biochem Biophys Acta, 1582(1-3):154-160; Rao et al., Pharmacological characterization of lysophospholipid receptor signal transduction pathways in rat cerebrocortical astrocytes, 2003, Brain Research, 990:182-194; Sorensen et al., Common signaling pathways link activation of murine PAR-1, LPA, and SIP receptors to proliferation of astrocytes, 2003, Molecular Pharmacology 64(5): 1199-1209).

### SUMMARY

The invention relates to lysophospholipid agents that have activity as modulators of lysophospholipid receptor activity. It has been surprisingly discovered that neuronal differentiation and neurogenesis may be modulated by a lysophospholipid agent, acting indirectly through astrocytes.

Methods embodying the underlining principles of the invention include methods of modulating neurite outgrowth, in culture or in a subject. The methods generally utilize cell-signaling agents which interact and bind to G protein-coupled cellular receptors (GPCRs). Such agents include phospholypid agents, especially lysophospholipid agents, such as lysophosphatidic acid (LPA) and sphingosine 1-phosphate (S1P). The methods include contacting astrocytes with an effective amount of a lysophospholipid agent, and contacting neurons with the astrocytes. In another aspect, the methods include treating neurons by contacting the neurons with astrocytes pretreated with a lysophospholipid agent. In a further aspect, the methods include contacting the neurons with an effective amount of an astrocyte-derived soluble factor (ADSF).

In another aspect, the methods embodying the principles of the invention include methods of treating a subject in which the methods include: identifying a subject in need of increased neurite outgrowth, and administering to the subject a lysophospholipid agent in an amount sufficient to increase neurite outgrowth, wherein the lysophospholipid agent is: (a) LPA, (b) an LPA analog; (c) an LPA derivative, e.g., a substituted LPA; (d) a LPA receptor agonist; (e) S1P; (f) a S1P analog; (g) a S1P receptor agonist; (h) a LPA-treated astrocyte: (i) a S1P-treated astrocyte; (j) a non-lysophospholipid that acts as an agonist; (k) a synthetic agonist; (l) an astrocyte-derived soluble factor (ADSF): or (m) combination of thereof. LPA and S1P receptor agonists include agents that are chemically distinct from lysophospholipids yet are biologically active.

In yet another embodiment, there are provided methods of treating pain, especially neuropathic pain, or multiple sclerosis (MS). The methods include administering to a subject in need an effective amount of a lysophospholipid agent.

The invention also embodies screening methods, i.e., methods of identifying agents that modulate neurite outgrowth. The methods include contacting astrocytes with a test agent; and co-culturing the astrocytes with neurons to determine neurite growth as compared to in the absence of the test agent. Such screening methods are used to identify lysophospholipid agonists or antagonists that may be chemically distinct from lysophospholipids, including small molecules.

Other advantages and a better appreciation of the specific adaptations, compositional variations, and physical and chemical attributes of the present invention will be gained upon an examination of the following detailed description of the invention, taken in conjunction with the accompanying drawings.

### BRIEF DESCRIPTION OF THE DRAWINGS

The invention may be better understood and appreciated by reference to the detailed description of specific embodiments presented herein in conjunction with the accompanying drawings of which:

FIGS. 1A-E illustrates increased neuronal commitment by LPA-treated astrocytes.

FIGS. 2 A-C illustrates LPA-treated astrocytes inducement of neuronal arborization.

FIGS. 3 A-G illustrates measurement of LPA-like activity in astrocyte conditioned medium.

FIGS. 4 A-F illustrates increased neuronal differentiation by conditioned medium derived from LPA-treated astrocytes.

FIGS. 5 A-B illustrates a soluble astrocyte derived factor increases neuronal differentiation.

FIGS. 6 A-C illustrates the soluble astrocyte derived factor can be heat inactivated.

FIGS. 7 A-D illustrates morphology and GRAP immunostaining of astrocytes from LPA₁(−/−LPA₂ (−/−) mice;

FIGS. 8 A-F illustrates effects of LPA on neurons mediated by LPA₁ and LPA₂ on astrocytes; and

FIG. 9 is a schematic model of LPA effect on neurons mediated by astrocytes.

FIG. 10A is a schematic of the retrovirus constructs containing null vector (SOO3, a), Ipa₁ (b), or Ipa₂ (c). FIGS. 10 B-G demonstrate the rescue of LPA₁ and LPA₂ effects on Ipa₁/Ipa₂ double-null mice by infection with the retroviral vectors for LPA₁ or LPA₂.

### DETAILED DESCRIPTION

The inventor has surprisingly found effects of lysophospholipid agents on cerebral neuronal differentiation that are mediated by astrocytes. An in vitro system of neuron-astrocyte co-culture was used to assess indirect effects of lysophospholipid agents, mediated by astrocytes, on cerebral cortical neuronal differentiation. Astrocytes treated with lysophospholipid agents increase neuronal fate commitment and neuritic arborization. Glial cells, thus, have a novel attribute as mediators of lysophospholipid effects on nervous system development and function, which also provides a new perspective on the role of astrocytes in nervous system disorders.

LPA and S1P receptors are widely distributed throughout CNS, both in neurons and glia; however, the precise role of astrocytic LPA and S1P receptors on neuronal development is unclear. The inventor has found that astrocytes previously treated with LPA provide a more permissive substrate for neurite outgrowth, which indicates a role of glial cells as mediators of LPA effects on neuronal differentiation within the embryonic cerebral cortex.

By using a co-culture system of cortical progenitors and cerebral cortical astrocytes, it has been demonstrated that astrocytes treated with LPA trigger neuronal fate commitment. The lack of LPA responses in astrocytes derived from LPA₁/LPA₂ double-null mice indicates that these effects are receptor-mediated. For the first time, in accordance with the invention, evidence is shown that astrocytes reconcile LPA actions and create a new scenario where LPA, or lysophospholipid agents generally, can be considered a novel mediator of neuron-astrocyte interaction during nervous system development and function.

Before any embodiments of the invention are explained in detail, it is to be understood that the invention is not limited in its application to the details of the structure and function set forth in the following description or illustrated in the appended drawings. The invention is capable of other embodiments and of being practiced or of being carried out in various ways. Further, no admission is made that any reference, including any patent or patent document, citied in this specification constitutes prior art. In particular, it will be understood that unless otherwise stated, reference to any document herein does not constitute an admission that any of these documents forms part of the common general knowledge in the art in the United States or in any other country. Any discussion of the references states what the author asserts and the applicant reserves the right to challenge the accuracy and pertinence of any of the documents cited herein.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless otherwise noted, technical terms are used according to conventional usage. Definitions of common terms in molecular biology may be found in references, such as Molecular Biology and Biotechnology: a Comprehensive Desk Reference, published by VCH Publishers, Inc., 1995 (ISBN 1-56081-569-8), Robert A. Meyers (ed.). However, as used herein, the following definitions may be useful in aiding the skilled practitioner in understanding the invention:

The term “treating” is meant to refer to reducing, diminishing, minimizing, controlling, alleviating or preventing a pathological condition or disorder, or the symptoms associated with a pathological condition or disorder, e.g., pain.

The terms “modulating” or “modulate” in connection with e.g., neurite outgrowth or neurogenesis is meant to refer to a change in neurite outgrowth or neurogenesis. For example, modulation may cause an increase or decrease in neuronal differentiation. Further, modulation may cause a change in interaction and binding to GPCRs. Most suitably, modulation of biological activity is to increase such activity. Suitably, the increase in activity is at least 10%, at least 20%, at least 30%, at least 50%, at least 60%, at least 70%, at least 80%, at least 100%, at least 200% relative to a suitable control.

As recognized by those of ordinary skill in the art, the term “effective amount” or “therapeutically effective amount” is meant to refer to an amount of an active agent, when administered to cells or a subject in need thereof is sufficient to produce a selected effect. For example, an effective amount of a lysophospholipid is an amount that increases the cell signaling activity of the lysophospholipid receptor.

The term “central nervous system” or “CNS” includes all cells and tissue of the brain and spinal cord of a vertebrate. Thus, the term includes, but is not limited to, neuronal cells, glial cells, astrocytes, cerebrospinal fluid (CSF), interstitial spaces and the like.

The term “glial cells” is meant to refer to various cells of the CNS also known as microglia, astrocytes, and oligodendrocytes.

As used herein, the term “LPA receptor” is meant to refer to cellular receptors that interact with LPA and other lysophospholipid agents, e.g., by binding and activation, to manifest physiological or pathophysiological effects of LPA. The LPA receptors that have been identified include LPA₁, LPA₂, LPA₃, LPA₄ and LPA₅, etc.

As used herein, the term “S1P receptor” is meant to refer to cellular receptors that interact with S1P or other lysophospholipid agents, e.g., by binding and activation, to manifest physiological or pathophysiological effects of S1P. The S1P receptors that have been identified include S1P₁, S1P₂, S1P₃, S1P₄ and S1P₅ etc.

As used herein, the term “lysophospholipid agent” is meant to refer to agents that bind to specific G protein-coupled receptors (GPCRs) and modulate, e.g., activate, certain signaling pathways, i.e., by inducing a detectable increase in receptor activity in vivo and in vitro (e.g., at least a 10% increase in receptor activity). Lysophospholipid agents include, but are not limited to, LPAs, LPA analogs, LPA derivatives, LPA receptor agonists, and other agents, which are sufficiently structurally or functionally similar to LPA to elicit a LPA receptor response, as well as S1P, S1P analogs, S1P derivatives, S1P receptor agonists, and other agents which are sufficiently structurally or functionally similar to S1P to elicit a S1P receptor response. In other words, the term “lysophospholipid agent,” in accordance with the invention includes any biologically active variants, analogs, mimetics, agonists, antagonists and derivatives. “Biologically active” in this context means having biological activity of a lysophospholipid, but it is understood that the activity of the variant analog, mimetics, agonist, antagonist or derivative thereof can be less potent or more potent than LPA or S1P. Further, agonists and antagonists and mimetics that function as agonists and antagonists include synthetic compounds specifically designed to mimic physiochemical properties of lysophospholipids, i.e., modulate, GPCRs, and can be chemically distinct from the lysophospholipid structure, including small molecules (as defined herein below). Lysophospholipid agents also include partial agonists and potentiators of LPA and S1P receptor activities. Many lysophospholipids are available commercially, e.g., from Avanti Polar Lipids, and many others are reported in the literature. Lysophospholipids are not limited to LPA and S1P (e.g., lysophosphatidyl choline, sphingosylphosphorylcholine, etc.), and there may be other receptors which could interact with these other lysophospholipids. It is also contemplated that targeted responses may be affected by using antibodies against the LPA and S1P receptors, particularly the LPA₁ receptor. Such antibodies can be made by methods known in the art.

The terms “analog” and “derivative” are used to refer to a molecule that structurally resembles a reference molecule but which has been modified to replace specific substituents on the reference molecule compared to the reference molecule. Analogs and derivatives are expected to have the same, similar, or improved utility. Syntheses and screening of analogs and derivatives having the desired properties can be accomplished through pharmaceutical chemical techniques.

The term “small molecule” as used herein is meant to refer to a composition, which has a molecular weight of less than about 5 kD, suitably less than about 4 kD. Small molecules include both organic (i.e., carbon-containing) and inorganic molecules.

The term “test agent” includes any substance, molecule, compound, entity, or a combination thereof. It includes, but is not limited to, e.g., protein, polypeptide, small molecule, polysaccharide, polynucleotide, and the like. It can be a natural product, a synthetic compound or a combination thereof.

Generally, lysophospholipid agents useful in accordance with the invention can be determined by employing certain assays which are standard and known to those skilled in the art, as noted in the citations below. For example, the assay set out in Hecht et al., Ventricular zone gene-1 (vgv-I) encodes a lysophosphatidic acid receptor expressed in neurogenic regions of the developing cerebral cortex, J. Cell Bio., 1996, 135:1071-1083, incorporated herein by reference, for LPA receptor agonists, which encompasses the use of ³H-LPA bound specifically to cells that overexpress or heterologously express the LPA receptor (see also Fukushima et al., 1998, A single receptor encoded by vzg-1/IpA1/edg-2 couples to G proteins and mediates multiple cellular responses to lysophosphatidic acid, PNAS, 95: 6151-6156, incorporated herein by reference). Other assays include the use of cell rounding or stress fiber formation in cells that do not express the receptor; once the receptor is heterologously expressed, these cells will then either round (in the case of the neuroblastoma cell line B103) or form stress fibers (for the liver cell line RH7777) when exposed to LPA at nM concentrations but not after exposure to related ligands. Another assay is to measure cAMP levels, since LPA activating its receptor produces a decrease in cAMP by activation of the heterotrimeric G-protein Gᵢ. Yet another way is to assay the proximal event in G protein coupling through the use of ³⁵S-GTPγS labeling of G proteins that is dependent on the presence of an LPA receptor and LPA stimulation or S1P and S1P receptor stimulation, respectively.

In the following description of embodiments of the methods of the invention, process steps are carried out at room temperature or 37° C., and atmospheric pressure unless otherwise specified. Standard techniques are used for cell culture, including CO₂%, with analyses also being standard and including fixing, staining, and immunostaining. The techniques and procedures are performed according to conventional methods in the art and various general references that are provided throughout this document. The procedures therein are well known in the art, some of which are provided for the convenience of the reader.

It also is specifically understood that any numerical value recited herein includes all values from the lower value to the upper value, i.e., all possible combinations of numerical values between the lowest value and the highest value enumerated are to be considered to be expressly stated in this application. All ranges disclosed herein encompass any and all possible subranges and combination of subranges. For example, if a concentration range is stated as 1% to 50%, it is intended that values such as 2% to 40%, 10% to 30%, or 1% to 3%, etc., are expressly enumerated in this specification. These are only examples of what is specifically intended. Also, all language such as “up to”, “at least”, “greater than”, “more than”, and the like include the number recited and refer to the ranges which can be subsequently broken down into subranges as discussed above. In the same manner, all ratios disclosed herein also include all subratios falling within the broader ratio.

In an illustrated embodiment, the invention embodies methods of modulating neuronal function, such as neurite outgrowth and neuronal differentiation, utilizing LPA receptor agonists. Particularly suitable are agonists of the LPA₁ receptor. Lysophospholipid agonists of the invention suitably activate the LPA receptor. Activators include agents that have agonist, partial agonist or potentiator activity at the LPA receptor as well as analogs of those compounds that have been modified to resist enzymatic modification or provide a suitable substrate of enzymatic conversion of an administered form into a more active form.

Phospholipids are generally represented by the general formula I:
P—X-L (I)
wherein P is a phosphate head group, X is a linker region and L is a lipophilic tail.

Specifically, LPAs are suitably represented by the general formula II: 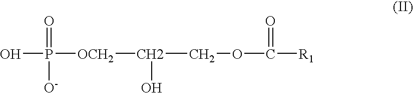
wherein R₁ is a C₁₅-C₂₅ saturated or unsaturated hydrocarbon chain.

LPA analogs, receptor agonists and antagonists that may be useful in accordance with the invention include those disclosed in, e.g., U.S. Pat. Nos. 7,169,818; 6,949,529; 6,380,177; 6,004,579; 5,565,439; and U.S. Published Application No. 2003/0027800, which are incorporated by reference in their entireties.

S1P, which has a structure related to LPA including a nitrogeneous base. S1Ps are suitably represented by the formula III: 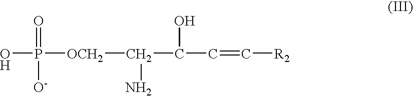
wherein R₂ is typically a C₁₋₃ hydrocarbon, but may be a longer saturated or unsaturated hydrocarbon chain.

S1P analogs and derivatives that may be useful in the methods embodying the principles of the invention include those disclosed in, e.g., U.S. Pat. No. 7,064,217, which is incorporated by reference in its entirety.

Neurite extension and retraction are important processes in the establishment of networks during development. Axonal navigation is largely orchestrated by a variety of guidance signals in the axons' surrounding environment. These cues include diffusible attractive or repellent molecules secreted by the intermediate or final cellular targets of the axons and extracellular matrix (ECM) components

As demonstrated in the examples below, conditioned medium of astrocytes treated with LPA mimics LPA effects on neuronal specification and neuritic arborization suggesting that these events involve soluble factors secreted by astrocytes in response to LPA signaling. Previous work demonstrated that LPA stimulates the expression of various cytokine genes in astrocytes such as nerve growth factor, interleukin-1 beta (IL-1), IL-3 and IL-6.

Although data of the examples below clearly implicate a soluble factor in this phenomenon, the involvement of ECM molecules cannot completely be ruled out. LPA and SIP have been demonstrated to enhance the binding and modulate the assembly of fibronectin on the surface of non-neural cells. Previous studies have associated the pattern of laminin deposition with astrocytic permissivity to neuritogenesis. The fact that LPA-conditioned medium also increases astrocyte permissivity to neuritogenesis strongly suggested that, if ECM modulation occurs, is likely to be due to a soluble factor secreted by astrocytes in response to LPA. The inventor has previously described a similar phenomenon: EGF induces neurite outgrowth of cerebellar neurons by modulating the content of laminin and fibronectin on astrocyte surface, thus enhancing cerebellar neuritogenesis in vitro.

It is noted that several previous works demonstrated that LPA induces neurite retraction, growth cone collapse and soma retraction in neuroblast primary culture and cerebral cortical neuroblast cells lines. The data described herein on the effect of LPA-astrocytes on neurite outgrowth are apparently in contrast to those obtained from direct action of LPA on axonal growth. However, all of these previous works deal with astrocyte-free cultures, which are devoid of any analysis of a putative astrocyte-mediated effect of LPA on neurogenesis.

As in the developing mammalian CNS, astrocytes constitute a major substratum for neuronal migration and axonal growth in the injured adult CNS. In the latter case, however, astrocytes are a key component of reactive gliosis, a major impediment to axonal regeneration. A considerable effort has been made over the last decades to understand the molecular mechanisms underlying this switch from a permissive to a non-permissive phenotype of astrocytes. Recently, activation of LPA receptors has been demonstrated to lead to astrogliosis in vivo and proliferation in vitro. Thus, whereas LPA induces astrogliosis characteristics, there are some data reporting its role on axonal growth. An LPA direct neuritogenic effect has been recently proposed. Fujiwara et al., 2003 demonstrated that cPA (cyclic phosphatidic acid), a LPA-analog, elicited a neurotrophic effect and promoted neurite outgrowth in cultured embryonic hippocampal neurons (Fujiwara et al., Cyclic phosphatidic acid elicits neurotrophin-like actions in embryonic hippocampan neurons, 2003, Journal of Neurochemistry, 87(5):1272-1283.).

Five cognate GPCRs have been shown to mediate the cellular effects of LPA in mammals; however, there is apparently receptor-specificity for each cellular response. The diversity of receptors and signaling pathways, sometimes leads to opposing responses such as rounding of cells stimulated by LPA₁ or LPA₂ versus the extension of neurites by LPA₃. Activation of different receptor isoforms can differently lead to activation of diverse pathways. Therefore, it is contemplated that LPA has a dual, antagonist effect on regeneration: 1) a harmful, astrogliosis-promoting effect with subsequent expression of growth inhibitory molecules and 2) a novel, axonal promoting activity due to modulation of expression of axonal growth molecules. This scenario is yet more complicated by emerging data pointing cross-communication between LPA and other growth factors such as PDGF (platelet derived growth factor), NGF (nerve growth factor) and TGF-β (transforming growth factor beta). Thus, a complex interplay between GPCRs and other family of receptors such as tyrosine and serine-threonine kinase receptors provides fine-tuning mechanisms for cellular response to lysophospholipids and might ultimately determine the final biological effects of these molecules. Understanding the specific pathways activated by LPA may lead to therapeutic advances in CNS injury treatment.

As contemplated in the schematic shown in FIG. 9 and described in the examples below, LPA serves as an extracellular signal from postmitotic neurons to proliferating neuroblasts and astrocytes. By acting through astrocyte LPA receptors, LPA induces secretion of a soluble factor(s), ADSF, which induces neuronal fate commitment and enhances neuronal maturation. The present data suggest that LPA is a novel mediator of neuron-astrocyte interaction during nervous system development and provides a new perspective in the understanding of astrocyte role in nervous system disorders.

In another embodiment, the methods of the principles of the invention are contemplated to be of value in treating pain, especially neuropathic pain, and multiple sclerosis. Such methods are generally accomplished by administering to a subject in need of treatment an effective amount of a lysophospholipid agent, e.g., an LPA, an LPA analog, an LPA receptor agonist, S1P, a S1P analog, a S1P receptor agonist, or a composition containing same, to prevent, reduce or otherwise diminish neuropathic pain, pain or multiple sclerosis. The methods can be used in any animal as a patient, and particularly, in any mammal, including, without limitation, primates, rodents, livestock and domestic pets. The methods are especially suitable to treat humans.

The invention is also encompassing pharmaceutical compositions including an effective amount of one or more lysophospholipids, receptor agonists and antagonists, and/or pharmaceutically acceptable excipients. For example, in accordance with the invention, an effective amount is an amount that when administered to neurons or to a subject would promote neurite growth and neuron differentiation.

As noted, the agents employed in the methods of the invention may be prepared in a number of ways well known to those skilled in the art. All preparations disclosed in association with the invention are contemplated to be practiced on any scale, including milligram, gram, multigram, kilogram, multikilogram or commercial pharmaceutical scale.

The particular mode of administration of the lysophospholipid agent selected will depend, of course, upon the particular lysophospholipid agent or combination of agents selected, the severity of the disease being treated, the general health condition of the patient, and the dosage required for therapeutic efficacy. The methods of this invention, generally speaking, may be practiced using any mode of administration that is medically acceptable, i.e., any mode that produces effective levels of the active compounds without causing clinically unacceptable adverse effects. Such modes of administration include oral, rectal, topical (as by powder, ointment, drops, transdermal patch or iontophoretic devise), transdermal, sublingual, intramuscular, infusion, intravenous, pulmonary, intramuscular, intracavity, as an aerosol, aural (e.g., via eardrops), intranasal, inhalation, or subcutaneous. Direct injection could also be used for local delivery. Oral or subcutaneous administration may be suitable for prophylacetic or long-term treatment because of the convenience of the patient as well as the dosing schedule.

Other delivery systems may include time-release, delayed-release or sustained-release delivery systems. Such systems can avoid repeated administrations of the compounds of the invention, increasing convenience to the patient and the physician and maintaining sustained plasma levels of compounds. Many types of controlled-release delivery systems are available and known to those of ordinary skill in the art. Sustained- or controlled-release compositions can be formulated, e.g., as liposomes or those wherein the active compound is protected with differentially degradable coatings, such as by microencapsulation, multiple coatings, etc.

For ease of administration, a pharmaceutical composition of the lysophospholipid or synthetic agonist may also contain one or more pharmaceutically acceptable excipients, such as lubricants, diluents, binders, carriers, and disintegrants. Other auxiliary agents may include, e.g., stabilizers, wetting agents, emulsifiers, salts for influencing osmotic pressure, coloring, flavoring and/or aromatic active compounds.

A pharmaceutically acceptable carrier or excipient refers to a non-toxic solid, semi-solid or liquid filler, dilutent, encapsulating material or formulation auxiliary of any type. For example, suitable pharmaceutically acceptable carriers, diluents, solvents or vehicles include, but are not limited to, water, salt (buffer) solutions, alcohols, gum arabic, mineral and vegetable oils, benzyl alcohols, polyethylene glycols, gelatin, carbohydrates such as lactose, amylose or starch, magnesium stearate, talc, silicic acid, viscous paraffin, vegetable oils, fatty acid monoglycerides and diglycerides, pentaerythritol fatty acid esters, hydroxy methylcellulose, polyvinyl pyrrolidone, etc. Proper fluidity may be maintained, for example, by the use of coating materials such as lecithin, by the maintenance of the required particle size in the case of dispersions and by the use of surfactants. Prevention of the action of microorganisms may be ensured by the inclusion of various antibacterial and antifungal agents such as paraben, chlorobutanol, phenol, sorbic acid and the like.

The dosage of active agent to be administered in accordance with the invention depends on the active agent selected; the disease or condition; the route of administration; the health and weight of the recipient; the existence of other concurrent treatment; if any, the frequency of treatment, the nature of the effect desired, for example, relief of pain; and the judgment of the skilled practitioner. The precise dose to be employed is decided according to the judgment of the practitioner and each patient's circumstances.

The level of active agent in a formulation can vary within the full range employed by those skilled in the art, e.g., from about 0.01 percent weight (% w) to about 99.99% w of the drug based on the total formulation and about 0.01% w to 99.99% w excipient. Generally, an acceptable daily dose is of about 0.001 to 50 mg per kilogram body weight of the recipient per day, preferably about 0.05 to 10 mg per kilogram body weight per day. Thus, for administration to a 70 kg person, the dosage range would be about 0.07 mg to 3.5 g per day, preferably about 3.5 mg to 1.75 g per day, and most preferably about 0.7 mg to 0.7 g per day depending upon the individuals and disease state being treated. Concentrations may range for the submicromolar to micromolar.

The lysophospholipid agents in accordance with the invention may also be co-administered with other therapeutic agents, e.g., other pain relieving agents, such as COX-2 inhibitors, such as celecoxib, rofecoxib, valdecoxib or parecoxib; 5-lipoxygenase inhibitors; low dose aspirin; NSAID's, such as diclofenac, indomethacin or ibuprofen; leukotriene receptor antagonists; DMARD's such as methotrexate; adenosine 1 agonists; recombinant human TNF receptor fusion proteins such as etanercept; sodium channel antagonists, such as lamotrigene; NMDA antagonists, such as glycine antagonists; and 5HT, agonists, such as triptans, for example sumatriptan, naratriptan, zolmitriptan, eletriptan, frovatriptan, almotriptan or rizatriptan.

The term “co-administration” is meant to refer to any administration route in which two or more agents are administered to a patient or subject. For example, the agents may be administered together, or before or after each other. The agents may be administered by different routes, e.g., one agent may be administered intravenously while the second agent is administered intramuscularly, intravenously or orally. The agents may be administered simultaneously or sequentially, as long as they are given in a manner sufficient to allow both agents to achieve effective concentrations in the body. The agents also may be in an admixture, as, for example, in a single tablet. In sequential administration, one agent may directly follow administration of the other or the agents may be given episodically. An example of a suitable co-administration regimen is where LPA is administered sequentially with a COX-2 inhibitor. When the lysophospholipids are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a lysophospholipid agent is used in combination with a second therapeutic agent active against the same medical condition, the dose of each compound may differ from that when the compound is used alone. The combination may also lead to a synergy where lower doses may be used than when the drugs are used alone.

In yet another embodiment, the invention embodies an astrocyte-derived soluble factor(s) (ADSF), pharmaceutical compositions thereof and methods utilizing ADSF. The methods include contacting neurons with ADSF. ADSF is derived from astrocytes treated with a lysophospholipid. Without being held to any particular theory, it is believed that astrocytes treated with a lysophospholipid agent, e.g., LPA, secrete a soluble factor that appears in the medium environment. This conditioned medium containing ADSF may then be used to treat neurons to elicit neurite outgrowth, differentiation and proliferation. In other words, the ADSF can be used directly to effect neuronal function.

In a still further embodiment, the invention also provides methods of identifying an agent that modulates neurite outgrowth. The methods include contacting astrocytes with a test agent; and co-culturing the astrocytes with neurons to determine neurite growth as compared to in the absence of the test agent. The method can screen either lysophospholipid agonist and antagonists.

Methods embodying the principles of the invention are further explained by the following examples, which should not be construed by way of limiting the scope of the present invention.

### EXAMPLES

### Example 1

### Astrocyte Primary Cultures

All animal protocols were approved by the Animal Research Committee of The Scripps Research Institute, conformed to National Institutes of Health guidelines and public law. Astrocyte primary cultures were prepared from cerebral cortex of newborn mice as previously described (de Sampaio e Spohr et al., Neuron-glia interaction effects on GFAP gene: a novel role for transforming growth factor-31, 2002, Eur J Neurosci, 16:2059-2069, incorporated herein by reference and Sousa et al., Glial fibrillary acidic protein gene promoter is differently modulated by transforming growth factor-beta 1 in astrocytes from distinct brain regions, 2004, Eur Neurosci, 19(7):1721-1730, incorporated by reference in its entirety). Astrocytes cultures were generated from C57B1/6 and Swiss mice. Briefly, after the mice were anesthetized, they were decapitated, brain structures were removed and the meninges were carefully stripped off. Dissociated cells were plated onto glass coverslips in 24 wells-plate (Corning Incorporated, NY), previously coated with polyornithine (1.5 μg/ml, mol. wt. 41,000, Sigma Chemical Co., St. Louis, Mo.), in DMEM/F12 medium supplemented with 10% fetal calf serum (Invitrogen, Carlsbad, Calif.). The cultures were incubated at 37° C. in a humidified 5% CO2, 95% air chamber for 10 days until reaching confluence. For experiments with LPA null mice, embryos from LPA, LPA₂ double-heterozygous females (on a mixed background C57B1/6×129SW) were genotyped by PCR using DNA isolated from a small part of the tail (Contos et al., Requirement of the LPA, lysophosphatidic acid receptor gene in normal suckling behavior, 2000, PNAS, 97(24):13384-13389, incorporated herein by reference; Contos et al., Characterization of LPA(2) (Edg4) and LPA(I)/LPA(2) (edg2/Edg4) lysophosphatidic acid receptor knockout mice: signaling deficits without obvious pehotypic abnormality attributable to LPA(2), 2002, Mol Cell Bio, 22(19):6921-6929, incorporated herein by reference).

### Example 2

### LPA Treatment and Conditioned Medium Preparation

After reaching confluence, glial mono layers were washed three times with serum-free DMEM/F12 medium and incubated as previously described for an additional day in serum-free medium. Cultures were then treated with 1 μM LPA (Oleoyl-LPA, Avanti Polar Lipids) in DMEM/F12 supplemented with 0.1% fatty-acid free bovine serum albumin (FAFBSA, Sigma) for 4 hours. Control astrocyte carpets were treated with DMEM/F12 supplemented with 0.1% FAFBSA. Medium was then replaced by DMEM/F12 without serum and used as substrate in neuron-astrocyte assays.

For astrocyte conditioned medium preparation, after astrocyte mono layers were treated with LPA-FAFBSA or FAFBSA, medium was replaced by DMEM-F12 and cultures were maintained for an additional day. CM derived from either LPA-treated astrocytes (LPA CM) or control cultures (Control CM) was recovered, centrifuged at 1500 g for 10 min, and used immediately or stored in aliquots at −70° C. for further use.

### Example 3

### Astrocyte-Neuron Co-Culture Assays

For neuronal cultures, timed-pregnant BALB/c females (Simonsen Laboratories), C57B1/6 females or Swiss females were killed by halothane followed by cervical dislocation, and embryos were removed at the day 14 (E14). Cortical progenitors were prepared from cerebral hemispheres from E14 embryos as previously described (Martinez and Gomes, 2002, Neuritogenesis induced by thyroid hormone-treated astrocytes is mediated by epidermal growth factor/mitogen-activated protein kinase-phosphatidylinositol 3-kinase pathways and involves modulation of extracellular matrix proteins, J Biol Chem, 277:49311-49318, incorporated herein by reference; Sousa et al., 2004, Glial fibrillary acidic protein gene promoter is differently modulated by transforming growth factor-beta 1 in astrocytes from distinct brain regions, 2004, Eur J Neurosci 19(7):1721-17302004, incorporated herein by reference). Briefly, cells were freshly dissociated from cerebral hemispheres and 1×10⁵ cells plated onto glial monolayer carpets non-treated or previously treated with LPA or LPA-conditioned medium for 4 hours as previously described. In the case of LPA CM assays, the medium was not replaced after 4 hours of treatment; instead it was left until the end of co-culture. Co-cultures were kept for 24 hours at 37° C. in a humidified 5% CO₂, 95% air atmosphere.

### Example 4

### Immunocytochemistry

Immunocytochemistry was performed as previously described (Martinez and Gomes, Neuritogenesis induced by thyroid hormone-treated astrocytes is mediated by epidermal growth factor/mitogen-activated protein kinase-phophatidylinositol 3-kinase pathways and involves modulation of extracellular matrix proteins, 2002, J Biol Chem, 277:49311-49318, incorporated herein by reference). Briefly, cultured cells were fixed with 4% paraformaldehyde (PFA) for 30 min and permeabilized with 0.2% Triton X-100 for 5 min at room temperature. For peroxidase assays, endogenous peroxidase activity was abolished with 3% H₂O₂ for 15 minutes followed by extensive washing with phosphate-buffered saline (PBS).

After permeabilization, cells were blocked with 10% normal goat serum (NGS, Vector Laboratories, Inc, Burlingame, Calif.) in PBS (blocking solution) for 1 hour, and incubated overnight at room temperature with the specified primary antibodies diluted in blocking solution. Primary antibodies were mouse anti-β-tubulin III antibody (Promega Corporation; Madison, Wis.; 1:1 000); rabbit anti-cleaved caspase-3 (Cell Signaling; Beverly, Mass.; 1:50); rabbit anti-GFAP (glial fibrillary acidic protein; Dako Corporation; Glostrup, Denmark; 1:200).

After primary antibody incubation, cells were extensively washed with PBSII, O % NGS and incubated with secondary antibodies for 1 hour, at room temperature. Secondary antibodies were: goat anti-mouse IgG conjugated with alexa fluor 488 (Molecular Probes, Eugene, Oreg.; 1:500); goat anti-rabbit IgG conjugated with alexa fluor 546 (Molecular Probes; Eugene, Oreg.; 1:500); anti-mouse IgG horseradish conjugated (Amersham Bioscience; Buckinghamshire, England; 1:200). Peroxidase activity was revealed with the Dako Cytomation kit (Liquid DAB and Chromo gem System). Negative controls were performed by omitting primary antibody during staining. In all cases no reactivity was observed when the primary antibody was absent. Cell preparations were mounted directly on N-propyl gallate and visualized by using a Nikon microscope. In case of the peroxidase reactions, cell preparations were dehydrated in a graded ethanol series, and mounted in entellan (Merck; Darm, Germany).

### Example 5

### Quantitative Analysis

To determine cell density, neuron number and cell death in different condition assays, neuron-astrocyte cocultures were labeled with DAPI (4′-6-Diamidino-2-phenylindole; Sigma-Aldrich; St Louis, Mo.) (total cells) and immunostained for the neuronal marker, class III β-tubulin or for the apoptosis marker, active caspase-3, respectively. Positive cells were visualized and counted using a Nikon microscope. At least five fields were counted per well. In all cases, at least 100 neurons randomly chosen were observed per well. The experiments were done in triplicate, and each result represents the mean of three independent experiments. Statistical analysis was done by ANOVA.

### Example 6

### Determination of LPA-Like Activity in Astrocyte CM

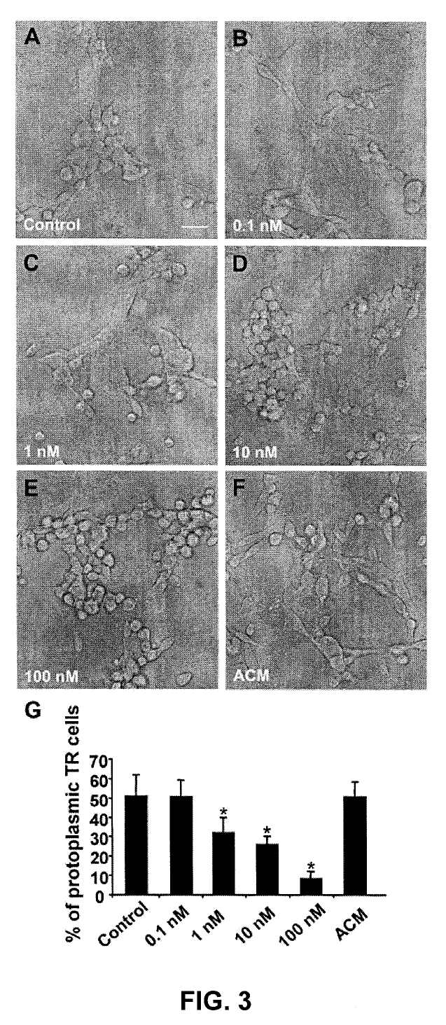

LPA-like activity was assayed by measuring morphological changes in TR mouse cerebral cortical immortalized neuroblast cells as previously described (Chun and Jaenisch, Clonal cell lines produced by infection of neocortical neuroblast using multiple oncogenes transduced by retroviruses, 1996, Mol Cell Neurosci, 18:379-383; Hecht et al., Ventricular zone gene-1 (vzg-I) encodes a lysophoshpatidic acid receptor expressed in neurogeneic regions of the developing cerebral cortex, 1996, J Cell Bio, 135:1071-1083, incorporated herein by reference; Ishii et al., Functional comparisons of the lysophosphatidic acid receptors, LPA(A1)IVZG-I/EDG-2, LP(A2)/EDG-4, and LP(A3)/EDG-7 in neuronal cell lines using a retrovirus expression system, 2000, Molecular Pharmacology, 58(5):895-902, incorporated herein by references; Fukushima et al., Lysophosphatidic acid influences the morphology and motility of young, postmitotic cortical neurons, 2002, Mol Cell Neurosci, 20(2):271-282, incorporated herein by reference). Cells were maintained in DMEM (Invitrogen, Carlsbad, Calif.) containing 10% fetal calf serum and penicillin/streptomycin. For experiments, cells were grown on (poly)lysine/coverslips for 24 hours in Opti-MEM I (Invitrogen, Carlsbad, Calif.) supplemented with 55 μM β-mercaptoethanol, 20 mM glucose, and penicillin/streptomycin. Before the assay, TR cells were serum starved overnight, and then cultivated with astrocyte conditioned medium (ACM) for 15-30 minutes. After this period, cells were fixed with 4% PFA and round cells were counted under phase-contrast optics (A-F). The concentration of LPA-activity in CM was estimated by comparison to a standard LPA dose-response curve (0.1 to 100 nM LPA). As shown in FIG. 3, ACM did not induces neurite retraction in TR cells, indicating that astrocytes do not secrete LPA under this condition (P<0.05). Scale bar in FIG. 3 corresponds to 50 μm.

### Example 7

### Astrocytes Previously Treated with LPA Enhance the Number of Neurons and Neuronal Arborization

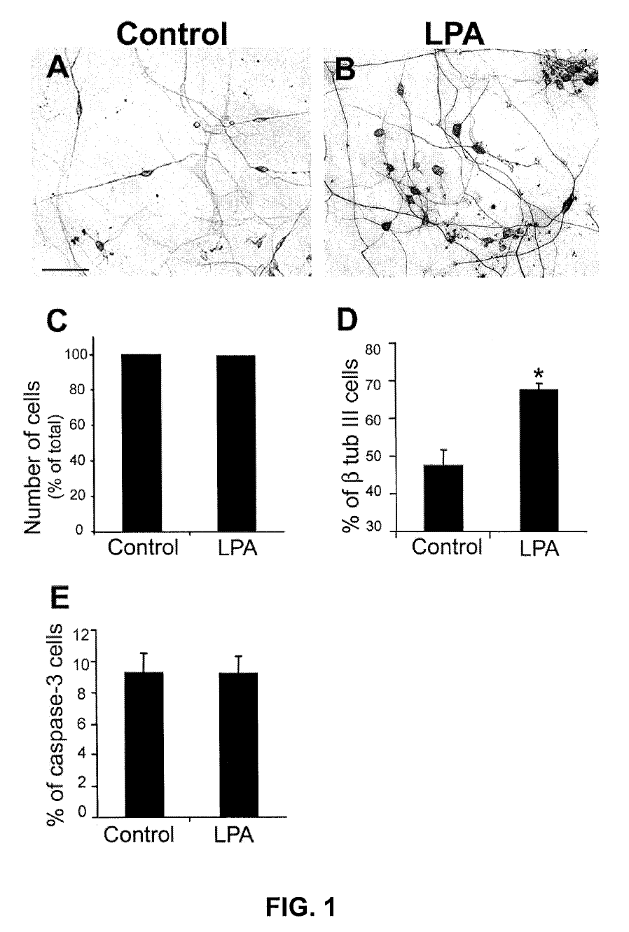

To investigate the role of astrocytes as mediators of LPA action in cerebral cortex ontogenesis, neuronal specification and number of neurites of cortical neurons cultivated onto astrocytes previously treated with LPA were analyzed. As shown in FIG. 1, cortical neuronal progenitors derived from 14-day embryonic mice (E14) were plated onto cortical astrocyte mono layers treated with LPA (B) and onto astrocytes treated with control (A) for 24 hours. After 24 hours, cells were fixed and immunostained using an antibody against the neuronal marker, β-tubulin III, and against the cell death marker, active caspase-3. Cell labeling was expressed as a percentage of the total cell number, revealed by DAPI staining. In all cases, at least 100 neurons randomly chosen were observed.

The total number of neurons and arborization of their neurites were measured. Such analysis revealed a clear difference between neurons plated on the two carpets. There was a 41% increase in the number of β-tubulin III positive cells plated onto LPA-treated astrocyte monolayers (FIG. 1D); in other words, LPA treatment of astrocytes indirectly enhanced neuronal specification.

To analyze the effect of astrocytes treated with LPA on neuronal survival, the number of cells expressing activated caspase-3 (a marker of apoptosis) after 24 hours of coculture was evaluated. As demonstrated in FIG. 1E, there was no difference in the number of caspase positive cells cultured either in control or treated cultures. The total number of cells was not altered by plating the progenitor cells onto LPA-astrocyte mono layers, which suggests that such LPA-astrocyte effect in neuronal number is mainly due to induction of neuronal fate commitment (FIG. 1E). For (C) and (E), P>0.05; for (D), P<0.05. Scale bar in FIG. 1 corresponds to 30 μm.

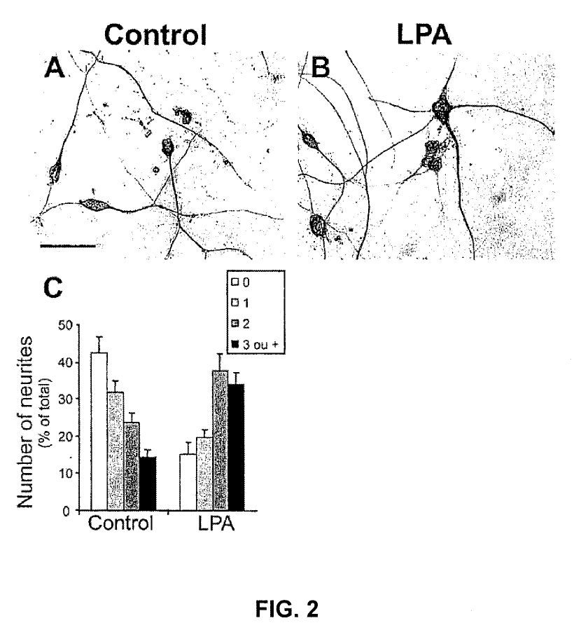

As shown in FIG. 2, neurons treated with LPA-treated astrocyte were morphologically characterized and the number of neurites evaluated (C). Analysis of neuronal morphology revealed a dramatic enhancement on the number of processes of neurons plated onto LPA-treated astrocytes. A significant increase was observed on the number of neurons with two neurites on LPA-treated astrocytes (FIG. 2C). Only a few neurons extended three or more neurites when plated onto control mono layers. On the other hand, a dramatic increase in this population was observed on LPA-treated cultures (FIG. 2C). A complex neuritic network was frequently observed on neurons plated onto LPA-astrocytes. Furthermore, as shown in FIG. 2, LPA treatment of astrocytes decreased by 64% the number of aneuritic neurons. Statistical significance was observed for all groups (P<0.05). The scale bar for FIG. 2 corresponds to 20 μm.

### Example 8

### Cerebral Cortical Astrocytes do not Secrete LPA in Culture

Postmitotic neurons have been reported to represent an endogenous source of LPA during nervous system development; however, other in vivo sources of extracellular signaling LPA in the nervous system are not completely known. Studies were set up to determine whether astrocytes from newborn mice could produce extracellular LPA. Because LPA is also produced during membrane biosynthesis, it was necessary to turn to a cell culture system in which hypothesized release of LPA into the medium could be discriminated from the LPA present in intracellular compartments.

To address this issue, a previously established bioassay based on heterologous expression of LPA receptors in TR mouse cerebral cortical immortalized neuroblast cells was used (Chun and Jaenisch, Clonal cell lines produced by infection of neocortical neuroblast using multiple oncogenes transduced by retroviruses, 1996, Mol Cell Neurosci, 18:379-383; Hecht et al., Ventricular zone gene-1 (vzg-1) encodes a lysophoshpatidic acid receptor expressed in neurogeneic regions of the developing cerebral cortex, 1996, J Cell Bio, 135:1071-1083, incorporated herein by reference; Ishii et al., Functional comparisons of the lysophosphatidic acid receptors, LPA(A1)IVZG-I/EDG-2, LP(A2)/EDG-4, and LP(A3)/EDG-7 in neuronal cell lines using a retrovirus expression system, 2000, Molecular Pharmacology, 58(5):895-902, incorporated herein by references; Fukushima et al., Lysophosphatidic acid influences the morphology and motility of young, postmitotic cortical neurons, 2002, Mol Cell Neurosci, 20(2):271-282, incorporated herein by reference). TR cells extend their bipolar or multipolar processes on glass coverslips under serum-free conditions. These cells express LPAj and LPAz and respond to LPA with rapid retraction of their processes resulting in cell rounding (Hecht et al., Ventricular zone gene-I (vzg-1) encodes a lysophosphatidic acid receptor expressed in neurogenic regions of the developing cerebral cortex 1996, J Cell Bio 135:1071-1083;; Ishii et al., Functional comparisons of the lysophosphatidic acid receptors, LPA(A1)IVZG-I/EDG-2, LP(A2)/EDG-4, and LP(A3)/EDG-7 in neuronal cell lines using a retrovirus expression system, 2000, Molecular Pharmacology, 58(5):895-902, incorporated herein by references).

As shown in FIG. 3, TR mouse cerebral cortical immortalized neuroblast cells were cultivated for 15-30 minutes in the presence of astrocyte conditioned medium (ACM). After this period, cells were fixed with 4% PFA and round cells were counted under phase-contrast optics (A-F) The cell number was expressed by percentage of protophasmic, non-round population. A LPA dose-response standard curve allowed estimation of the LPA concentrations in the conditioned medium. Addition of concentrations raging from 1 to 100 nM of LPA induced rounding of TR cells (FIG. 3). By contrast, ACM did not induce neurite retraction in TR cells suggesting that astrocytes do not secrete LPA under these conditions, i.e., LPA-like activity is absent in this medium (FIG. 3).

### Example 9

### LPA-Astrocyte Induced Neurogenesis and Neuritogenesis Involves an Astrocytic Soluble Factor

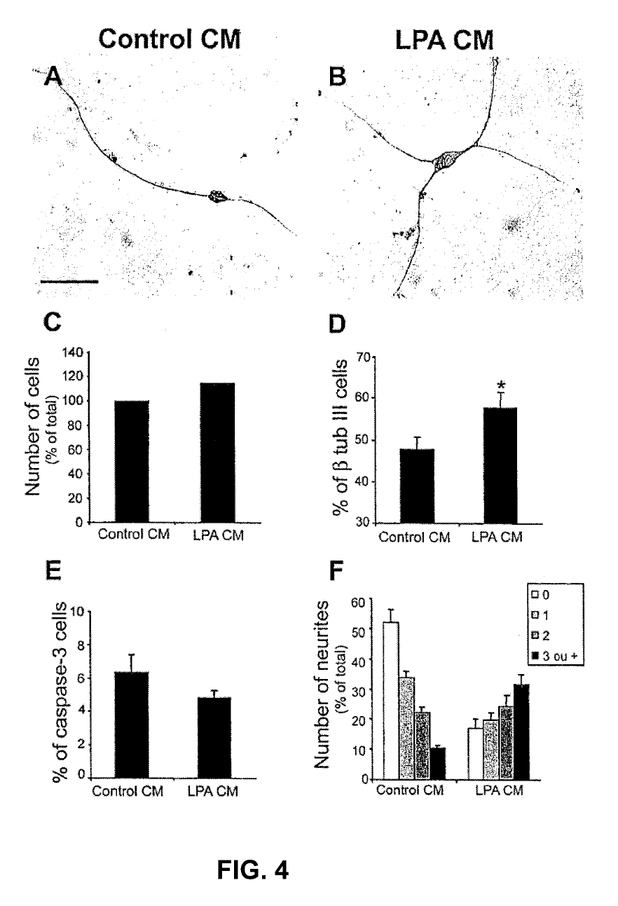

To evaluate the involvement of LPA-astrocyte derived soluble factors on neurite outgrowth and neuronal specification, cerebral cortex astrocyte cultures were treated with conditioned medium derived from LPA-treated astrocytes (ACM), instead of with LPA itself. In this experimental paradigm, neither astrocytes nor neurons are in direct contact with LPA. Embryonic progenitors were cultured onto different astrocyte carpets in the presence of control conditioned medium (Control CM) or conditioned medium derived from LPA-treated astrocytes (ACM). The cells were fixed and immunostained as described above, and the number of neurons and neurite arborization were analyzed (FIG. 4A).

Treatment of neuron-astrocyte cocultures by ACM induced an increase in neuronal population although smaller than LPA treatment (FIG. 4D). Quantitative analyzes revealed that under this condition (ACM) there was a significant increase in the number of neurites. The fraction of aneuritic neurons was significantly decreased by LPA CM treatment (67%, FIG. 4F), whereas neurons with 3 or more processes were substantially increased (210%).

Neuronal death was not affected by ACM treatment of astrocytes as previously observed for LPA treatment. Number of active caspase-3 positive cells was not altered by plating progenitors cells onto ACM-treated carpets (FIG. 4E). The data indicate that conditioned medium derived from LPA treated astrocytes mimics the effects of LPA, suggesting that soluble factors secreted by astrocytes in response to LPA treatment are implicated in neuronal differentiation. Statistical significance for total cell number (C) and cell death (E) was P>0.05; for (D) and (F), P<0.05. The scale bar for FIG. 4 corresponds to 20 μm.

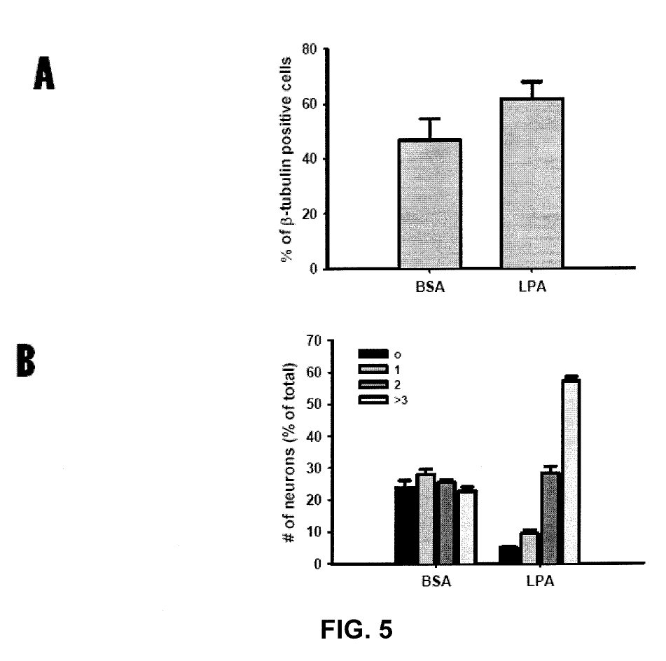

To further test the effects of soluble factors form LPA-treated astrocytes on neuronal differentiation, astrocytes were treated with LPA for 4 hours, media was changed and the astrocytes were incubated with neuronal progenitor cells that were on the top membrane in a Boyden chamber. Cells were cultivated for 24 hours, and the cells were fixed and immunostained as described above. As seen in FIG. 5, a soluble factor that traversed the Boyden chamber membrane was able to increase neuronal differentiation.

### Example 10

### Astrocytic Soluble Factor Produced from LPA or S1p-Treated Astrocytes is Heat Sensitive

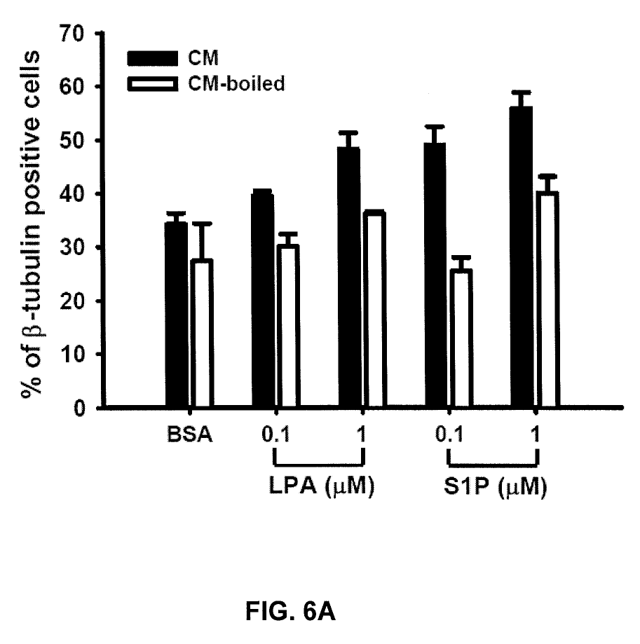

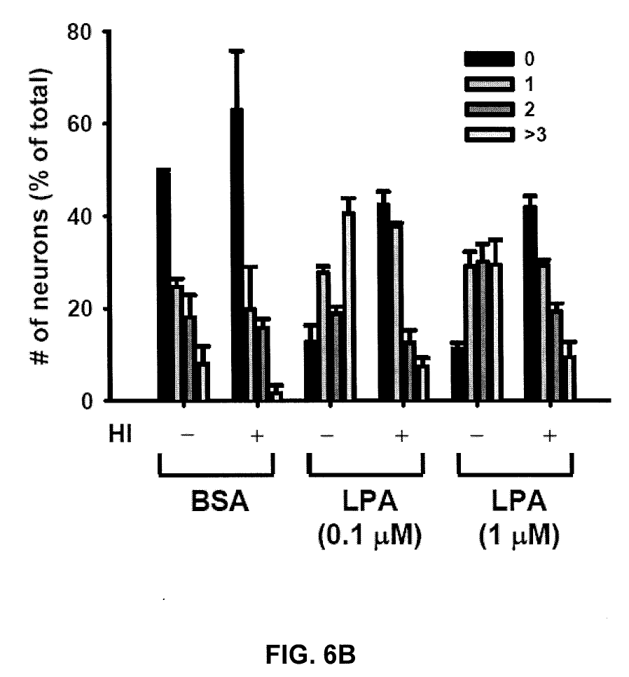

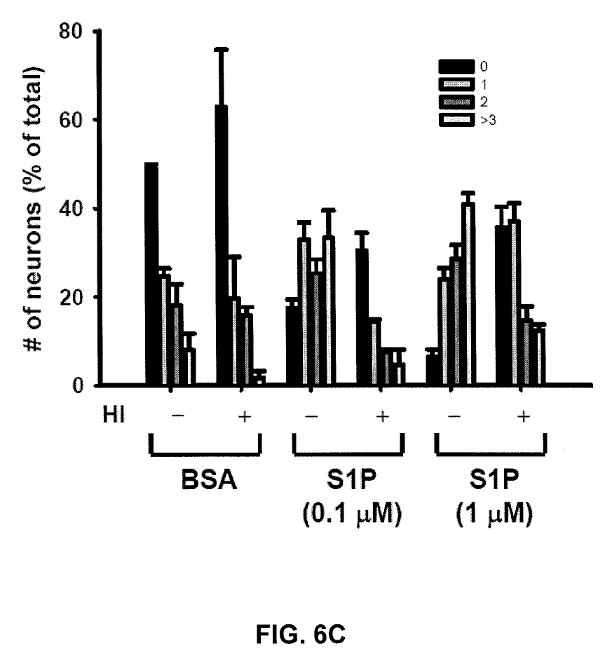

To determine some characteristics of the LPA-produced astrocytic soluble factor, astrocytes were treated with 0.1 μM or 1 μM LPA or S1P or BSA for 4 hours. Media was changed and cells were incubated for 24 hours at which time conditioned media (CM) was obtained. The CM was divided and half was heat inactivated by boiling for 30 min at 100° C. Neuronal progenitor cells (E13.5) were incubated for 24 hours with LPA-treated astrocyte CM or heat-inactivated CM, full strength replacement of diluents thereof, for 24 hours. The neuronal progenitor cells were fixed and immunostained as described above. As seen if FIG. 6A, the ability of the soluble factor produced from LPA- and S1P-treated cells to cause neuronal differentiation is inactivated by heat-inactivation of the CM. As seen if FIGS. 6B and 6C, heat-inactivation (HI) of the LPA-treated astrocyte CM reduced the ability of the CM to elicit neuronal differentiation.

### Example 11

### LPA Effects on Neurons are Specifically Mediated by LPA, and LPA₂ Receptors on Astrocytes

The Generation of Receptor-Null Mice Allows not Only Direct Examination of the systemic roles of LPA receptors in vivo (Contos et al., Requirement of the LPA₁ lysophosphatidic acid receptor gene in normal suckling behavior, 2000, PNAS, 97(24):13384-13389, incorporated herein by reference; Contos et al., Characterization of LPA(2) (Edg4) and LPA(I)/LPA(2) (edg2/Edg4) lysophosphatidic acid receptor knockout mice: signaling deficits without obvious pehotypic abnormality attributable to LPA(2), 2002, Mol Cell Bio, 22(19):6921-6929, incorporated herein by reference) but it also contributes for further elucidation of LPA receptor-specific signaling pathways in receptor-null primary cells (Ishii et al., Functional comparisons of the lysophosphatidic acid receptors, LP(A1))/VZG-1/EDG-4, and LP(A3)/EDG-7 in neuronal cell lines using a retrovirus expression system, 2000, Mol. Pharmacology, 58:895-902, incorporated herein by reference). To determine whether LPA effects in the co-culture system are mediated by specific LPA receptors, astrocyte mono layers derived from mice with null mutations in both LPA₁ and LPA₂ receptors were prepared. Astrocyte primary cultures were prepared from cerebral cortex of wild type and LPA double-null newborn mice. Astrocyte mono layers were kept in DMEM/F12 medium supplemented with 10% fetal calf serum for days until reaching confluence. After this period, cultures were maintained in serum free medium and treated with 1 μM of LPA for 24 hours. Subsequently the cells were fixed and immunostained using an antibody against an astrocyte maturation marker, GFAP.

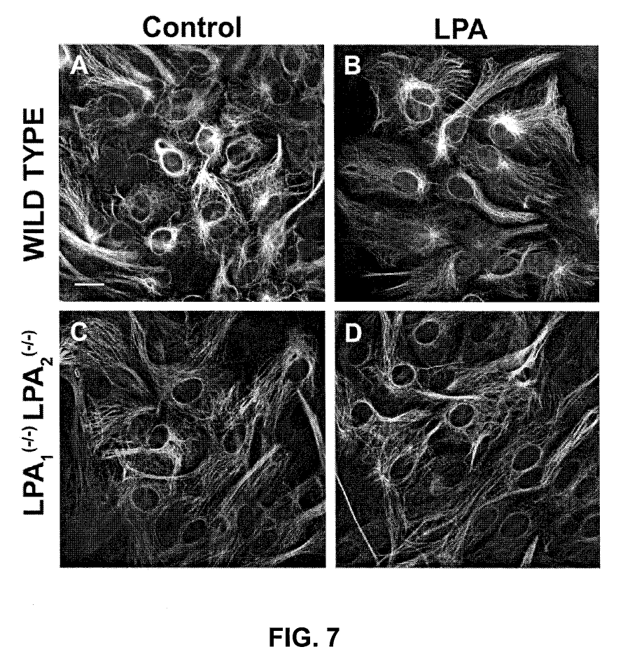

Morphological analyses did not reveal any obvious difference between wild type and LPA, LPA₂ null mice. Astrocyte derived from both mice present an intense labeling for of GFAP with a great network of intermediate filament extending from the perinuclear region through out the entire cytoplasm (FIGS. 7A;7C). Treatment of these cultures with 1 μM LPA did not affect astrocyte morphology (FIGS. 7B;7D). The scale bar in FIG. 7 corresponds to 50 μm.

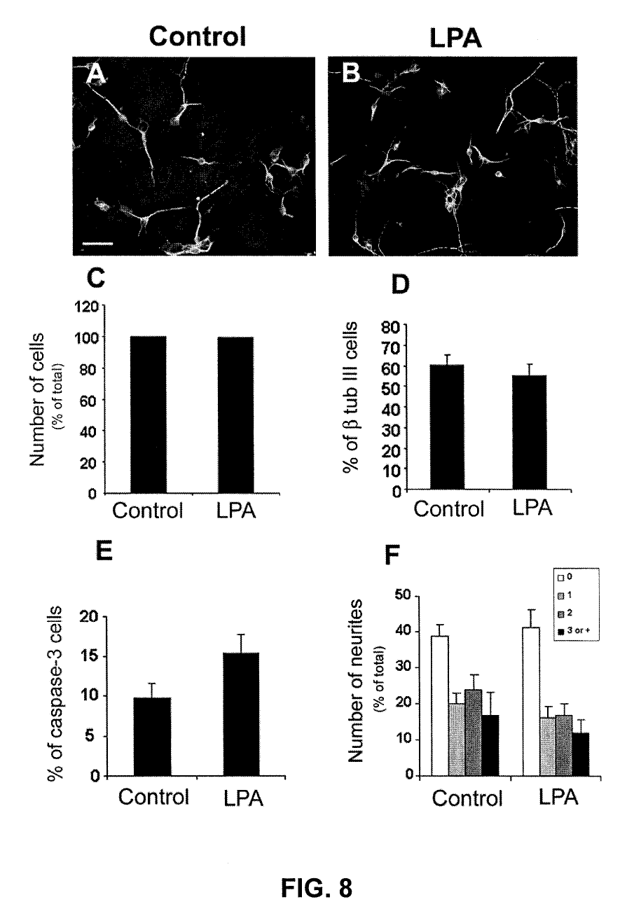

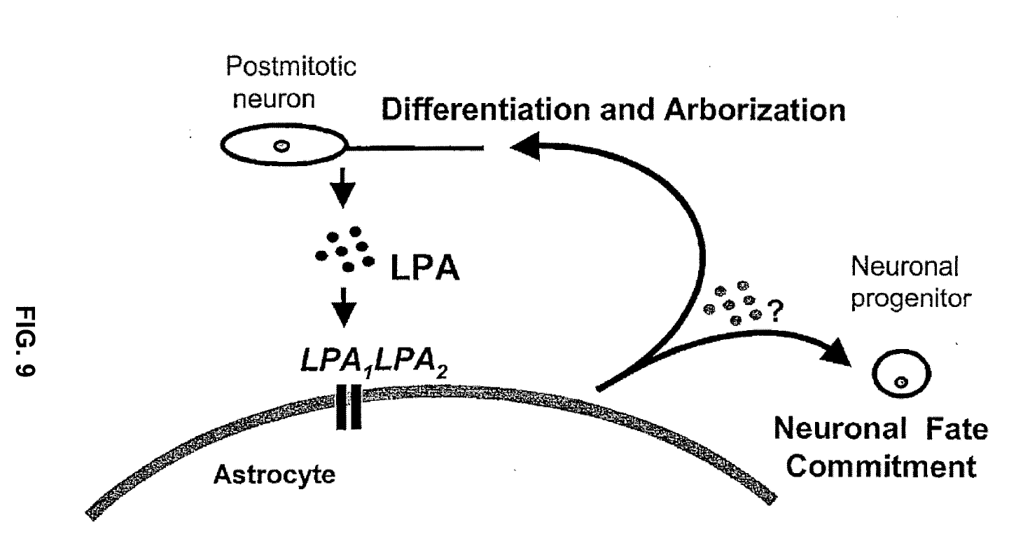

In order to address the involvement of LPA receptor in LPA-astrocyte effects on neuronal morphogenesis, cortical neuronal progenitors derived from E14 wild type mice were plated onto cortical astrocyte mono layers derived from LPA, LPA₂ null mice previously treated with LPA. After 24 hours, cells were immunostained for the neuronal marker, β-tubulin III, and the number of neurons and arborization of their neurites were measured. As shown in FIG. 8, treatment of these cell carpets with LPA did not affect neuronal population in contrast to wild type astrocytes treated with LPA, i.e., LPA-astrocyte mediated effects are absent in astrocytes derived from LPA₁ LPA₂ null mice. Neuronal death did not differ either in treated or non-treated astrocytes as previously shown for wild type astrocytes. P>0.05 for all situations shown in FIG. 8. The scale bar in FIG. 8 corresponds to 50 μm.

To further demonstrate that the observed effects seen after LPA treatment were due to both astrocyte expression of defined LPA receptors and LPA signaling and not a deficiency produced by LPA receptor deletion, a retroviral rescue strategy was utilized. Retroviral vectors expressing LPA₁ or LPA₂ were reported previously (Ishii et al., Functional comparisons of the lysophosphatidic acid receptors, LP(A1)/VZG-1/EDG-4, and LP(A3)/EDG-7 in neuronal cell lines using a retrovirus expression system, 2000, Mol. Pharmacology, 58:895-902, incorporated herein by reference) as depicted in FIG. 8A. LPA₁/LPA₂ double-null astrocytes were infected with the epitope-tagged LPA₁, LPA₂, or empty-vector control retrovirus in 4 μg/ml of polybrene to the media of subconfluent proliferating astrocytes plated in a monolayer. Plates were centrifuged (700 g) at 28° C. for 2 hours, and the astrocytes cultured for 48 hours in fresh media. Astrocytes were serum starved for another 24 hours and then used in the assays. Receptor expression was confirmed by epitope-tagged immunolabeling of GFP-positive cells.

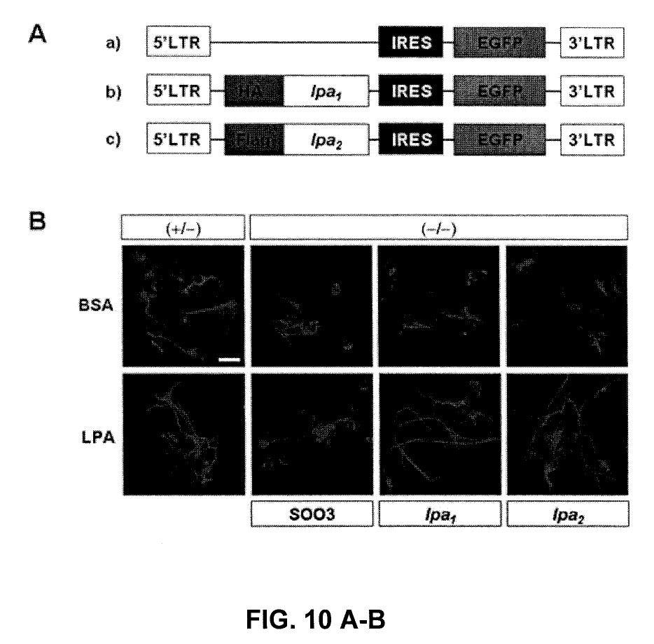

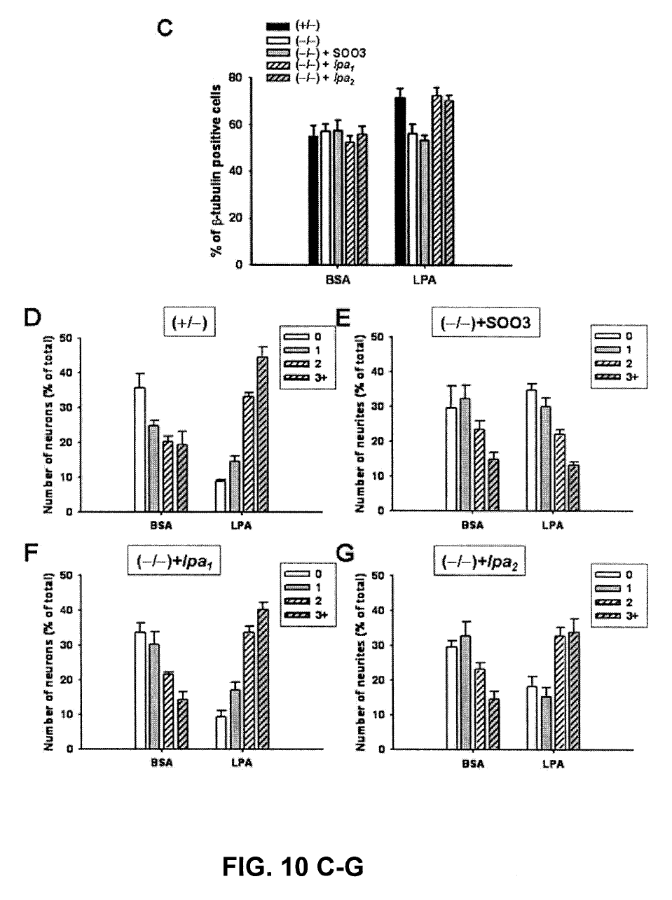

The retroviral infected LPA₁/LPA₂ double-null astrocytes were treated with LPA. The astrocytes monolayers were co-cultured with cortical neuronal progenitors derived from E14 wild type mice. After 24 hours, cells were immunostained for the neuronal marker, β-tubulin III, and the number of neurons and arborization of their neurites were measured. Priming of LPA₁/LPA₂ double-null astrocytes infected with the empty vector control virus did not result in an increase neuronal differentiation as seen in FIG. 10B, SOO3. In marked contract, double-null astrocytes infected with either the LPA₁ or LPA₂ retrovirus demonstrated increased β-tubulin III cells or increased prevalence of greater than two neurites/neurons, restoring LPA response patterns to levels that approximate those seen in wild-type controls for most neurite/neuron classes as seen in FIG. 10B-G. This data demonstrates that at lest partial rescue of LPA responsiveness in mutant astrocytes can be seen by re-expression of a single LPA receptor subtype.

Taken together, the findings herein indicate that the LPA-astrocyte effects observed here are specific and receptor-mediated. For the first time, an indirect action of LPA on neurogenesis/neuronal differentiation, mediated by astrocytes has been demonstrated.

### Example 12

### Treatment of Neuropathic Pain

The chronic constriction injury (CCI) model is used to induce the neuropathic hypersensitivity (Bennett & Xie, A peripheral mononeuropathy in rat that produces disorders of pain sensation like those seen in man, 1988, Pain, 33(1): 87-107, incorporated herein by reference) in rats. Under isoflurane anaesthesia, the common left sciatic nerve is exposed at mid thigh level and four loose ligatures of chromic gut are tied around it. The wound is then closed and secured using suture clips. The surgical procedure is identical for the sham-operated animals except the sciatic nerve is not ligated. The rats are allowed a period of seven days to recover from the surgery before behavioral testing began. An Isyophospholipid is dosed chronically for 14 days (days 20-33 postoperative). A reversal of the CCI-induced decrease in paw withdrawal threshold is seen following 3 days of chronic dosing which is maximal after 1 week. This reversal is maintained throughout the remainder of the dosing period. Following cessation of the drug treatment, the paw withdrawal threshold returns to that of the vehicle treated CCI-operated animals.

### Example 13

### Clinical Observations

A double-blind multicenter clinical trial for treatment of neuropathic pain is designed to assess the safety and efficacy of lysophospholipids or related lysophospholipid receptor agonists in accordance with the present invention. Patients are randomized to an active agent or placebo. Patients are monitored for perception and/or presence of pain using standard methods.

While the present invention has now been described and exemplified with some specificity, those skilled in the art will appreciate the various modifications, including variations, additions, and omissions that may be made in what has been described. Accordingly, it is intended that these modifications also be encompassed by the present invention and that the scope of the present invention be limited solely by the broadest interpretation that lawfully can be accorded the appended claims.

All patents, publications, references and data cited herein are hereby fully incorporated by reference. In case of conflict between the present disclosure and incorporated patents, publications, references and data, the present disclosure should control.

1. A method of modulating neurite outgrowth, comprising contacting astrocytes with an effective amount of a lysophospholipid agent, and contacting neurons with the astrocytes.

2. The method of claim 1, wherein the lysophospholipid agent is selected from the group consisting of an LPA, an LPA analog, an LPA derivative, an LPA receptor agonist, an LPA receptor antagonist, an S1P, a S1P analog, a S1P derivative, a S1P receptor agonist and a S1P receptor antagonist.

3. The method of claim 2, wherein the lysophospholipid agent is LPA.

4. The method of claim 2, wherein the lysophospholipid agent is S1P.

5. A method of promoting neurite outgrowth, comprising contacting neurons in culture or in a subject with an effective amount of an astrocyte-derived soluble factor (ADSF).

6. The method of claim 5, wherein there is an increase of neurite outgrowth is at least 10%.

7. The method of claim 5, wherein there is an increase of neurite outgrowth is at least 20%.

8. The method of claim 5, wherein there is an increase of neurite outgrowth is at least 30%.

9. A method of treating pain, comprising administering to a subject in need an effective amount of a lysophospholipid agent.

10. The method of claim 9, wherein the lysophospholipid agent is selected from the group consisting of an LPA, an LPA analog, an LPA derivative, an LPA receptor agonist, S1P, a S1P analog, a S1P derivative, and a S1P receptor agonist.

11. The method of claim 10, wherein the lysophospholipid agent is LPA.

12. The method of claim 10, wherein the lysophospholipid agent is S1P.

13. The method of claim 9, further comprising co-administering to a subject in need an effective amount of a therapeutic agent.

14. The method of claim 13, wherein the therapeutic agent is selected from the group consisting of a COX-2 inhibitor, a NSAID, a DMARD, a human TNF receptor fusion protein, a sodium channel antagonist, a NMDA antagonist, and a 5HT antagonist.

15. A method of identifying an agent that modulates neurite growth, comprising: contacting astrocytes with a test agent; and co-culturing the astrocytes with neurons to determine neurite growth as compared to in the absence of the test agent.

16. The method of claim 1, wherein the LPA receptor agonist is identified by the method of claim 15.

17. The method of claim 1, wherein the S1P receptor agonist is identified by the method of claim 15.

18. The method of claim 16, wherein the LPA receptor agonist is a small molecule

19. The method of claim 17, wherein the S1P receptor agonist is a small molecule.

20. A method for increasing neurite outgrowth, comprising exposing astrocytes to a lysophospholipid agent; preparing a conditioned medium from the astrocytes, and contacting neurons to the conditioned medium.

21. The method of claim 20, wherein the lysophospholipid agent is LPA.

22. The method of claim 20, wherein the lysophospholipid agent is a S1P.

23. The method of claim 20, wherein the lysophospholipid is selected from the group consisting of an LPA analog, an LPA derivative, an LPA receptor agonist, a S1P analog, a S1P derivative, and a S1P receptor agonist.

24. A method of modulating neurite outgrowth, comprising:
a) pretreating astrocytes with a lysophospholipid agent, and
b) contacting neurons with the astrocytes under conditions sufficient to modulate
neurite outgrowth.

25. The method of claim 24, wherein the modulating is an increase in neurite outgrowth.

26. Thee method of claim 24, wherein the neurons are in vitro.

27. The method of claim 24, wherein the neurons are in a subject.

28. The method of claim 24, wherein the lysophospholipid agent is LPA.

29. The method of claim 24, wherein the lysophospholipid agent is S1P.

30. The method of claim 24, wherein the lysophospholipid agent is selected from the group consisting of an LPA analog, an LPA derivative, an LPA receptor agonist, an LPA receptor antagonist, a S1P analog, a S1P derivative, a S1P receptor agonist and a S1P receptor antagonist.

31. A method of modulating neurite outgrowth, comprising contacting neurons in culture or in a subject with a medium conditioned by treatment of astrocytes with a lysophospholipid agent.

32. The method of claim 30, wherein the lysophospholipid agent is LPA.

33. The method of claim 30, wherein the lysophospholipid agent is S1P.

34. The method of claim 30, wherein the lysophospholipid agent is selected from the group consisting of an LPA analog, an LPA derivative, an LPA receptor agonist, an LPA receptor antagonist, a S1P analog, a S1P derivative, a S1P receptor agonist and a S1P receptor antagonist.

35. A method of treating a subject, the method comprising: identifying a subject in need of increased neurite outgrowth, and administering to the subject a lysophospholipid in an amount sufficient to increase neurite outgrowth, wherein the lysophospholipid is: (a) an LPA, (b) an LPA analog; (c) an LPA receptor agonist; (d) an LPA-treated astrocyte; (e) a S1P; (f) a S1P analog; (g) a S1P receptor agonist; (h) a S1P-treated astrocyte (i) an astrocyte-derived soluble factor (ADSF); or (j) combination thereof.

36. The method of claim 34, wherein the subject is a human.

37. The method of claim 34, wherein the subject has a CNS neuropathological condition.

38. The method of claim 36, wherein the condition is multiple sclerosis or neuropathic pain.

39. The method of claim 34, wherein the LPA receptor agonist is a small molecule identified by the method of claim 15.
